(19) United States
(12) Patent Application Publication
(10) Pub. No.: US 2026/0357318 A1
(43) Pub. Date: Nov. 12, 2026

(54) METHODS FOR MICROBIAL DETECTION

(71) Applicant: JANSSEN PHARMACEUTICA NV

(72) Inventors: James HAUSCHILD, Raritan, NJ (US); Scott WEISS, Burleson, TX (US)

(21) Appl. No.: 19/122,560

(22) PCT Filed: Oct. 20, 2023
(86) PCT No.: PCT/IB2023/060637

§ 371 Date: Apr. 18, 2025

Related U.S. Application Data
(60) Provisional application No. 63/380,407, filed on Oct. 21, 2022.

(51) Int. Cl. C12Q 1/02; C12Q 1/18; G01N 21/64

(52) U.S. Cl. CPC C12Q 1/025; C12Q 1/18; G01N 21/6428; G01N 21/6458; G01N 2021/6439

(57) ABSTRACT

Provided is a method for detecting a microbe in a sample comprising detecting confluence of the sample. Also provided is a method for assaying a test agent comprising adding the test agent to a sample comprising a microbe, and measuring confluence of the sample. In some embodiments, the detecting is automated. In some embodiments, the detecting is continuous. In some embodiments, the confluence is percent confluence.

### CROSS REFERENCE TO RELATED APPLICATIONS

This application is entitled to priority under 35 U.S.C. § 119(e) to U.S. Provisional Application No. 63/380,407, filed Oct. 21, 2022, which is hereby incorporated by reference in its entirety.

### FIELD

The general inventive concepts relate to the field of microbial detection and more particularly to methods for the automated detection of microbes by direct imaging.

### BACKGROUND

Conventional microbiological testing is based on 100 year-old technology using agar plates for enumerating microbial colony-forming units (CFU) or the visual detection of turbidity in liquid media, which is indicative of microbial growth. The conventional plate count for microbial bioburden monitoring of in-process or finished product samples requires 3-7 days of incubation for quantitative enumeration. Sterility testing is performed according to reference standards set forth by the United States Pharmacopeial Convention (USP). Non-sterile product bioburden testing is performed according to USP <61> Microbiological Examination of Nonsterile Products: Microbial Enumeration Tests and USP<62> using either membrane filtration (0.45 μm), plate-count methods or the most-probable number method. The total aerobic microbial count (TAMC) is determined using Soybean-Casein Digest Agar following 3-5 days incubation at 30 to 35 °C. The total combined yeasts and molds count (TYMC) is determined using Sabouraud Dextrose Agar following 5-7 days incubation at 20 to 25 °C. In contrast, the presence/absence sterile product finished product sterility test following USP<71> requires 14 days of incubation before visual confirmation of microbial growth. In USP<71> the sterile product is tested according to the minimum volumes specified and using the minimum number of samples articles specified in the standard. To remove any potential antimicrobial properties the product is filtered through a membrane filter (0.45 μm) to retain microorganisms, washed with a diluent to remove any antimicrobial components, and then incubated with media to allow for microbial growth. The apparatus is designed so that the solution to be tested can be introduced and filtered under aseptic conditions, allowing for the membrane to be aseptically transferred to medium, or is suitable for carrying out the incubation after adding the medium to the apparatus itself, such as performed with the commercial Steritest system. For sterile products without any antimicrobial properties direct inoculation into the medium can be performed. USP<71> states that sterility test media can be used provided that they comply with the requirements of the Growth Promotion Test of Aerobes, Anaerobes, and Fungi. The compendial media found to be suitable for the test for sterility are Fluid Thioglycollate Medium (FTM), primarily intended for the culture of anaerobic bacteria but will also detect aerobic bacteria, and Soybean-Casein Digest Medium (Tryptic Soy Broth (TSB)), suitable for the culture of both fungi and aerobic bacteria. The FTM and TSB samples are incubated at 14 days at 30 to 35 °C and 20 to 25 °C respectively and then visually inspected for the presence of turbidity, which in indicative of microbial growth. With the introduction of Cell Therapy Products, a USP informational chapter <1071> Rapid Sterility Testing of Short-Life Products: A Risk-Based Approach has been recently published.

For medical devices terminally sterilized with ionizing irradiation an incremental dose verification or sterilization dose substantiation is performed following ISO 11137. After exposure to a designated irradiation dose a test of sterility is performed on the medical device or medical device sample item portion (SIP). The irradiated medical device sample is aseptically transferred into TSB, incubated for 7 days at ~30 °C and then visually inspected for turbidity, which is indicative of microbial growth.

Conventional microbiology methods are manual, provide slow retrospective results, are open to subjective interpretation, have limited data traceability and have the potential for data integrity issues. Recent advancements have been made in the development of Alternative and Rapid Microbiological Methods (ARMM) to address these limitations, including but not limited to the development of CFR Part 11 compliant computer-based detection technologies such as colorimetric CO₂ detection, solid-phase laser scanning cytometry, flow cytometry, ATP bioluminescence, and automated detection of microcolony growth based on auto-fluorescent detection. However, all these detection technologies require unique sample preparation and, in some cases, specialized reagents for the rapid detection of microorganisms, requiring costly and time-consuming revalidation efforts prior to use. In addition, the surrogate detection of viable microorganisms without the direct determination of microbial growth with some of these alternative detection technologies can be destructive tests which sometimes yield false positive results, which is unacceptable for certain industrial microbiology applications such as finished product sterility testing or in-process microbial screening. There remains a need for methods for the automated detection of microbes by direct imaging.

### SUMMARY

Provided is a method for detecting a microbe in a sample comprising: detecting confluence of the sample.

In some embodiments, the confluence is detected using an image-based metric. In some embodiments, the image-based metric comprises direct imaging. In further embodiments, the direct imaging is direct cell imaging.

In some embodiments, the sample is in an incubator. In some embodiments, the detecting comprises detecting using a microscope or image sensor.

In some embodiments, the confluence of the sample is compared to the confluence of a control sample. In some embodiments, the confluence of the sample is monitored over a period of time. In some embodiments, the confluence of the sample is monitored continuously.

In some embodiments, the sample has been contacted with an antibody to the microbe.

In some embodiments, the sample is contacted with a fluorescent agent or a bioluminescent agent.

In some embodiments, the system further comprises detecting fluorescence of the sample. In further embodiments, the means for detecting fluorescence is a fluorimeter.

In some embodiments, the system further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, or Pseudomonas aeruginosa. In some embodiments, the microbe is Cutibacterium acnes, Staphylococcus aureus, Aspergillus brasiliensis, Candida albicans, Bacillus subtilis, Escherichia coli, or Pseudomonas aeruginosa.

In some embodiments, any genus or species of viable microbe may be detected.

In some embodiments, the detecting confluence is automated.

In some embodiments, the confluence is percent confluence.

Provided is a method for assaying a test agent comprising: adding a test agent to a sample comprising a microbe; and detecting confluence of the sample.

In some embodiments, the confluence is detected using an image-based metric.

In some embodiments, the image-based metric comprises direct imaging. In further embodiments, the direct imaging is direct cell imaging.

In some embodiments, the sample is in an incubator. In some embodiments, the detecting comprises detecting using a microscope or image sensor.

In some embodiments, the confluence of the sample is compared to the confluence of a control sample. In some embodiments, the confluence of the sample is monitored over a period of time. In some embodiments, the confluence of the sample is monitored continuously.

In some embodiments, the test agent is a compound, a supplement, an antibiotic, a bacteriophage, or a combination thereof.

In some embodiments, the method further comprises identifying the test agent as a prebiotic when the confluence of the sample is greater than the confluence of the control sample. In some embodiments, the method further comprises identifying the test agent as a prebiotic when the percent confluence of the sample is greater than the percent confluence of the control sample.

In some embodiments, the method further comprises identifying the test agent as a prebiotic when the confluence of the sample increases over a period of time.

In some embodiments, the method further comprises identifying the test agent as an antimicrobial agent when the confluence of the sample is less than the confluence of the control sample.

In some embodiments, the method further comprises identifying the test agent as an antimicrobial agent when the confluence of the sample decreases over a period of time.

In some embodiments, the sample has been contacted with an antibody to the microbe.

In some embodiments, the sample is contacted with a fluorescent agent or a bioluminescent agent.

In some embodiments, the system further comprises detecting fluorescence of the sample. In further embodiments, the means for detecting fluorescence is a fluorimeter.

In some embodiments, the system further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, or Pseudomonas aeruginosa. In some embodiments, the microbe is Cutibacterium acnes, Staphylococcus aureus, Aspergillus brasiliensis, Candida albicans, Bacillus subtilis, Escherichia coli, or Pseudomonas aeruginosa.

In some embodiments, any genus or species of viable microbe may be detected.

In some embodiments, the detecting confluence is automated.

In some embodiments, the confluence is percent confluence.

### DESCRIPTION OF THE FIGURES

FIGS. 1A-1C illustrate the IncuCyte System. FIG. 1A shows IncuCyte System Automated Direct Imaging vs Automated BacT/ALERT System CO₂ Colorimetric Detection Microbial Time-to-Detection Results Table. FIG. 1B shows IncuCyte Cutibacterium acnes Time-to-Detection. The time course shows a change in % confluence over time. FIG. 1C shows IncuCyte time course images of C. acnes proliferation.

FIG. 2 illustrates IncuCyte CAR-T Microbial Screening. The graph shows a time course showing changes in confluence across different groups. CAR-T samples were spiked with about 5 or 55 CFU of S. aureus (SA) in a 24-well microplate. Parallel TSB media only samples spiked with about 5 or 55 CFU SA were included in the 24-well microplate. Phase contrast imaging, 20 x, 36 images/well.

FIG. 3 illustrates CHO cell Bacillus subtilis spore spiking % confluence detection. The graph shows a time course showing detection of CHO cells contaminated with Bacillus subtilis.

FIG. 4 shows Staphylococcus epidermidis (SE) Prebiotic and Antimicrobial Screening (1%). Ingredient A (sterile filtered), SE Prebiotic activity was detected by faster % confluence growth compared to positive control. Ingredient B (sterile filtered), SE Antimicrobial activity was detected by no change in % confluence growth over time. Ingredient C (sterile filtered), SE Prebiotic activity was detected by faster % confluence growth compared to positive control.

FIG. 5 shows a Natural Product Extract Staphylococcus aureus (SA) Antimicrobial Screening (0.1%). 96-well Natural Product Extract Microplate % Confluence Results Map. Natural Product Extract SA Antimicrobial activity was detected by no change in % confluence growth over time for sample wells A3, E6, B9, C11 and D11.

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

“Confluence” as used herein is based on the amount of a culture vessel surface area that appears covered by cells, as compared to the total surface area of the culture vessel. The cells may be of any origin. In some embodiments, the cells are mammalian, insect, microbial, or combinations thereof. In some embodiments, confluence is percent confluence. In some embodiments, confluence may be measured on an image of a culture vessel. In some embodiments, the image is of a portion of a culture vessel. In some embodiments, confluence is based on the amount of an image surface area that appears covered by cells, as compared to the total surface area of the image. Confluence may be measured or calculated or may be determined by an algorithm. Confluence may be determined using IncuCyte. Confluence may be measured over a period of time. Initial confluence is measured at time zero. Confluence may be measured continously, or at time intervals, to determine confluence over time.

“Percent confluence” or “% confluence” as used herein is based on the percentage of a culture vessel surface area that appears covered by cells, as compared to the total surface area of the culture vessel. The cells may be of any origin. In some embodiments, the cells are mammalian, insect, microbial, or combinations thereof. In some embodiments, percent confluence may be measured on an image of a culture vessel. In some embodiments, the image is of a portion of a culture vessel. In some embodiments, confluence is based on the amount of an image surface area that appears covered by cells, as compared to the total surface area of the image. Percent confluence may be measured or calculated or may be determined by an algorithm. Percent confluence may be determined using IncuCyte. Percent confluence may be measured over a period of time. Initial percent confluence is measured at time zero. Percent confluence may be measured continously, or at time intervals, to determine percent confluence over time.

The terms “antibody” and “antibodies” as used herein are meant in a broad sense and include immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a domain antibody (dAb) fragment, which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated antibody specifically binding CD38 is substantially free of antibodies that specifically bind antigens other than human CD38). An isolated antibody that specifically binds CD38, however, can have cross-reactivity to other antigens, such as orthologs of human CD38, such as Macaca fascicularis (cynomolgus monkey) CD38. Moreover, an isolated antibody may be substantially free of other cellular material and/or chemicals.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A human antibody may also contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, sequences, or antibodies that are generated in vitro using Fab arm exchange such as bispecific antibodies.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The term “chimeric antigen receptor” or “CAR” as used herein means a synthetic or recombinant receptor comprising an antigen specific domain, a costimulatory domain and an intracellular signaling domain. In some embodiments, the CAR further comprises an extracellular hinge or spacer region, a transmembrane domain, or combinations thereof. In some embodiments, the antigen specific domain is an scFv.

The term “chimeric antigen receptor T cell” or “CAR-T” as used herein means a T cell expressing a CAR.

In some embodiments of any of the compositions or methods described herein, a range is intended to comprise every integer or fraction or value within the range.

Embodiments described herein as “comprising” one or more features may also be considered as disclosure of the corresponding embodiments “consisting of” and/or “consisting essentially of” such features.

To date the use of automated direct microscopic imaging for presence/absence microbial screening of non-sterile product or in-process samples, presence/absence testing of finished product sterility test samples, bioprocess microbial contamination monitoring, antibiotic or bacteriophage susceptibility testing or for prebiotic and antimicrobial product development applications have not been proposed. Provided herein are methods for the automated, non-invasive, non-destructive direct microscopic imaging of in-process and final product non-sterile and sterility test samples prepared following previously validated methods while providing significantly quicker time-to-detection than manual visible turbidity or the formation of colony-forming units (CFU) on agar growth media. In some embodiments, the methods utilize a 21 CFR Part 11 compliant system for data traceability and increased data integrity. The methods described herein allow for the use of direct microscopic imaging for bioprocess microbial contamination monitoring from aseptically collected bioreactor samples, antibiotic or bacteriophage susceptibility testing in clinical settings and for prebiotic and antimicrobial product development applications.

Provided is a method for the automated non-destructive and non-invasive detection of microbes by direct imaging in liquid media samples for increased data integrity and faster time-to-detection compared to conventional manual visual detection based on media turbidity or the formation of CFU on agar growth media. In some embodiments, the detection is continuous. Automated imaging systems such as the Essen BioScience IncuCyte system have a limit of detection of 10 μm for enumeration and are routinely used for mammalian cell imaging for enumeration and viability assessment applications. To overcome this 10 μm size limitation which prevents the direct detection and enumeration of microorganisms in the 1 μm size range the present inventors discovered that the mammalian cell imaging system's % confluence detection metric can be used for the rapid presence/absence determination for final product sterility testing, presence/absence screening of non-sterile product or in-process microbial screening, bioprocess microbial contamination monitoring, antibiotic or bacteriophage susceptibility testing and prebiotic ingredient and/or antimicrobial product development applications.

### Methods

Provided is a method for detecting a microbe in a sample comprising: detecting confluence of the sample.

In some embodiments, the confluence is percent confluence.

In some embodiments, the confluence is detected using an image-based metric. In some embodiments, the image-based metric comprises direct imaging. In further embodiments, the direct imaging is direct cell imaging.

In some embodiments, the sample is in an incubator. In some embodiments, the sample is under controlled temperature incubation. In some embodiments, the detecting comprises detecting using a microscope or image sensor.

In some embodiments, the sample is at a temperature from about 20 °C to about 40 °C. In some embodiments, the temperature is from about 20 °C to about 37 °C. In some embodiments, the temperature is from 25 °C to about 35 °C. In further embodiments, the temperature is from about 20 °C to about 25 °C. In yet further embodiments, the temperature is from 30 °C to about 35 °C.

In some embodiments, the sample is grown for about 0.1 day to about 7 days prior to measuring the confluence of the sample. In some embodiments, the sample is grown for about 0.25 day to about 7 days prior to measuring the confluence of the sample. In some embodiments, the sample is grown for about 0.25 days prior to measuring the confluence of the sample. In some embodiments, the sample is grown for about 0.5 days prior to measuring the confluence of the sample. In some embodiments, the sample is grown for about 1 day prior to measuring the confluence of the sample. In some embodiments, the sample is grown for about 2 days prior to measuring the confluence of the sample. In some embodiments, the sample is grown for about 3 days prior to measuring the confluence of the sample. In some embodiments, the sample is incubated for about 4 days prior to measuring the confluence of the sample. In some embodiments, the sample is incubated for about 5 to about 7 days prior to measuring the confluence. In some embodiments, the sample is incubated for about 5 days prior to measuring the confluence of the sample. In some embodiments, the sample is incubated for about 6 days prior to measuring the confluence of the sample. In some embodiments, the sample is incubated for about 7 days prior to measuring the confluence of the sample.

In some embodiments, the confluence of the sample is compared to the confluence of a control sample. In some embodiments, the confluence of the sample is monitored over a period of time.

In some embodiments, the sample has been contacted with an antibody to the microbe.

In some embodiments, the sample is contacted with a fluorescent agent or a bioluminescent agent.

In some embodiments, the system further comprises detecting fluorescence of the sample. In further embodiments, the means for detecting fluorescence is a fluorimeter.

In some embodiments, the system further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, or Pseudomonas aeruginosa. In some embodiments, the microbe is Cutibacterium acnes, Staphylococcus aureus, Aspergillus brasiliensis, Candida albicans, Bacillus subtilis, Escherichia coli, or Pseudomonas aeruginosa.

In some embodiments, any genus or species of viable microbe may be detected.

In some embodiments, the confluence of the sample is monitored over a period of time. In further embodiments, the confluence of the sample is monitored at set intervals, over a period of time. In yet further embodiments, the confluence of the sample is monitored at random intervals, over a period of time. In yet further embodiments, the confluence of the sample is monitored continuously, over a period of time. The period of time may be from about 0.1 to about 1000 hours, from about 0.1 to about 100 hours, from about 0.1 to about 72 hours, from about 0.1 to about 48 hours, from about 0.1 to about 24 hours, from about 0.1 to about 12 hours and from about 0.1 to about 6 hours.

In some embodiments, the detecting confluence is automated.

### Cells

In some embodiments, the sample comprises a eukaryotic cell. In further embodiments, the eukaryotic cell produces a therapeutic product. The therapeutic product may be released into the cell media, where it may be collected. The methods provided herein allow for detection of contamination.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell includes but is not limited to a mouse cell, a CHO cell, a T cell, or a B cell. In some embodiments, the mouse cell is a mouse Sp2/0 cell. In some embodiments, the eukaryotic cell is HEK293F. In some embodiments, the eukaryotic cell is PER.C6.

In some embodiments, the eukaryotic cell is a chimeric antigen receptor T cell (CAR-T cell).

In any of the methods described herein, the confluence may be percent confluence.

### Assays

Provided is a method for assaying a test agent comprising: adding a test agent to a sample comprising a microbe; and detecting confluence of the sample.

In some embodiments, the confluence is percent confluence.

In some embodiments, the confluence is detected using an image-based metric.

In some embodiments, the image-based metric comprises direct imaging. In further embodiments, the direct imaging is direct cell imaging.

In some embodiments, the sample is in an incubator. In some embodiments, the sample is under controlled temperature incubation. In some embodiments, the detecting comprises detecting using a microscope or image sensor.

In some embodiments, the sample is at a temperature from about 20 °C to about 40 °C. In some embodiments, the temperature is from about 20 °C to about 37 °C. In some embodiments, the temperature is from 25 °C to about 35 °C. In further embodiments, the temperature is from about 20 °C to about 25 °C. In yet further embodiments, the temperature is from 30 °C to about 35 °C.

In some embodiments, the sample is grown for about 0.1 day to about 7 days prior to measuring the confluence of the sample. In some embodiments, the sample is grown for about 0.25 day to about 7 days prior to measuring the confluence of the sample. In some embodiments, the sample is grown for about 0.25 days prior to measuring the confluence of the sample. In some embodiments, the sample is grown for about 0.5 days prior to measuring the confluence of the sample. In some embodiments, the sample is grown for about 1 day prior to measuring the confluence of the sample. In some embodiments, the sample is grown for about 2 days prior to measuring the confluence of the sample. In some embodiments, the sample is grown for about 3 days prior to measuring the confluence of the sample. In some embodiments, the sample is incubated for about 4 days prior to measuring the confluence of the sample. In some embodiments, the sample is incubated for about 5 to about 7 days prior to measuring the confluence. In some embodiments, the sample is incubated for about 5 days prior to measuring the confluence of the sample. In some embodiments, the sample is incubated for about 6 days prior to measuring the confluence of the sample. In some embodiments, the sample is incubated for about 7 days prior to measuring the confluence of the sample.

In some embodiments, the confluence of the sample is compared to the confluence of a control sample. In some embodiments, the confluence of the sample is monitored over a period of time. In some embodiments, the percent confluence of the sample is compared to the percent confluence of a control sample. In some embodiments, the percent confluence of the sample is monitored over a period of time.

In some embodiments, the test agent is a compound, a supplement, an antibiotic, a bacteriophage, or a combination thereof.

In some embodiments, the method further comprises identifying the test agent as a prebiotic when the confluence of the sample is greater than the confluence of the control sample. In some embodiments, the method further comprises identifying the test agent as a prebiotic when the percent confluence of the sample is greater than the percent confluence of the control sample.

In some embodiments, the method further comprises identifying the test agent as a prebiotic when the confluence of the sample increases over a period of time.

In some embodiments, the method further comprises identifying the test agent as an antimicrobial agent when the confluence of the sample is less than the confluence of the control sample. In some embodiments, the method further comprises identifying the test agent as an antimicrobial agent when the percent confluence of the sample is less than the percent confluence of the control sample.

In some embodiments, the method further comprises identifying the test agent as an antimicrobial agent when the confluence of the sample decreases over a period of time. In some embodiments, the method further comprises identifying the test agent as an antimicrobial agent when the percent confluence of the sample decreases over a period of time.

In some embodiments, the sample has been contacted with an antibody to the microbe.

In some embodiments, the sample is contacted with a fluorescent agent or a bioluminescent agent.

In some embodiments, the system further comprises detecting fluorescence of the sample. In further embodiments, the means for detecting fluorescence is a fluorimeter.

In some embodiments, the system further comprises detecting bioluminescence of the sample. In further embodiments, the means for detecting bioluminescence is a luminometer.

In some embodiments, the microbe includes but is not limited to Cutibacterium acnes, Staphylococcus aureus, Aspergillus brasiliensis, Candida albicans, Bacillus subtilis, Escherichia coli, or Pseudomonas aeruginosa. In some embodiments, the microbe is Cutibacterium acnes, Staphylococcus aureus, Aspergillus brasiliensis, Candida albicans, Bacillus subtilis, Escherichia coli, or Pseudomonas aeruginosa.

In some embodiments, any genus or species of viable microbe may be detected.

In some embodiments, the detecting confluence is automated.

### Cells

In some embodiments, the sample comprises a eukaryotic cell. In further embodiments, the eukaryotic cell produces a therapeutic product. The therapeutic product may be released into the cell media, where it may be collected. The methods provided herein allow for detection of contamination.

In some embodiments, the eukaryotic cell produces a protein, an antibody or fragment thereof, a duobody, a receptor, a chimeric antigen receptor, a glycoprotein, a viral vector, or a combination thereof.

In some embodiments, the eukaryotic cell includes but is not limited to a mouse cell, a CHO cell, a T cell, or a B cell. In some embodiments, the mouse cell is a mouse Sp2/0 cell. In some embodiments, the eukaryotic cell is HEK293F. In some embodiments, the eukaryotic cell is PER.C6.

In some embodiments, the eukaryotic cell is a chimeric antigen receptor T cell (CAR-T cell).

In any of the assays described herein, the confluence may be percent confluence.

### EXAMPLES

### Example 1: IncuCyte System Time Course

### Materials and Methods

### IncuCyte System Automated Direct Imaging vs Automated BacT/Alert System

BioBall™ samples (bioMerieux, USA) of designated microorganisms were resuspended following manufacturer's instructions. Separate time-to detection experiments were performed by inoculating approximately 25 CFU (colony-forming units) into either aerobic or anaerobic BacT/ALERT sample bottles or Tryptic soy broth (TSB) and Fluid Thioglycollate Medium (FTM) for BacT/ALERT system or IncuCyte system analysis respectively. Most of the test microorganisms were incubated aerobically using TSB based media, while only Cutibacterium acnes was incubated anaerobically in an anaerobic BacT/ALERT bottle containing TSB based media under anaerobic headspace conditions or FTM. Healthy T cell donor CAR-T cells (1E4-1E6) were present in all microorganism spiked samples except for the C. acnes IncuCyte sample which was FTM only. The C. acnes FTM only inoculum count was also less than 25 CFU for the IncuCyte system experiment as described below.

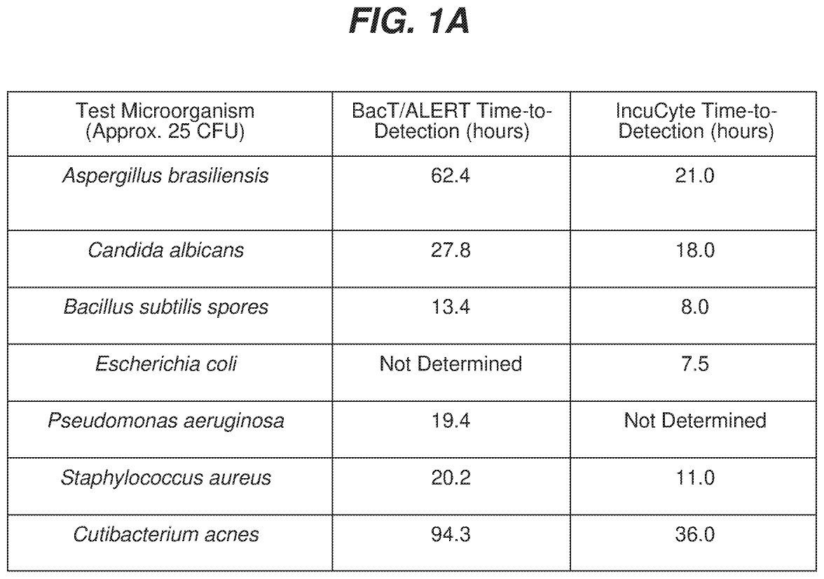

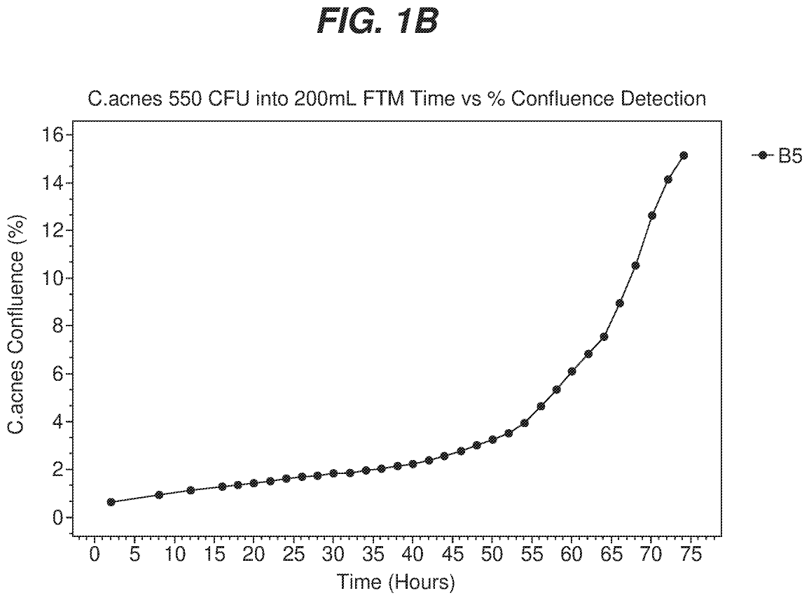

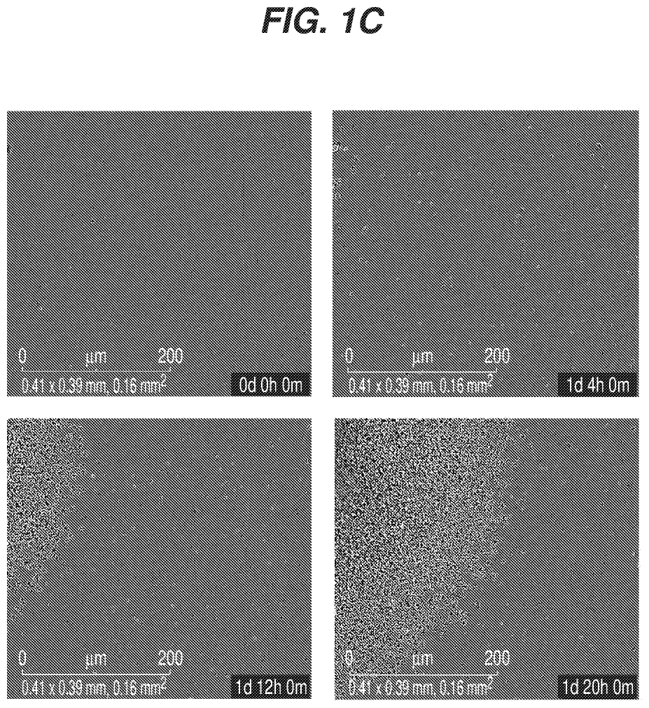

Twelve-well cell culture plates (plasma treated) containing approximately 6 mL of media in each well were used for all test microorganism IncuCyte experiments except for C. acnes which was carried out in a 24-well plate (plasma treated) containing approximately 3 mL of media. The BacT/ALERT system time-to-detection of positive microbial growth was indirectly determined based on colorimetric CO₂ detection algorithms. In contrast, the IncuCyte system time-to-detection of positive microbial growth was determined by direct microscopic imaging based on an increase in the % confluence metric over time for all test microorganisms. FIG. 1A shows the results of the separate BacT/ALERT and IncuCyte time-to-detection experiments.

Incucyte Cutibacterium acnes Time-to-Detection

A time course was carried out showing a change in % confluence over time. A 200 mL bottle of FTM was spiked with approximately 3 CFU/mL of C. acnes and then aseptically transferred (~70 mL) into a 24-well microplate (~3 mL/well). The sample plate was placed into the IncuCyte system inside 37 °C Incubator. Phase contrast imaging, 20 x, 36 images/well was performed every two hours. FIG. 1B shows the increase in the IncuCyte % confluence metric over time for C. acnes growth in FTM.

Incucyte Time Course Images of C. acnes Proliferation

As described above the IncuCyte system was set up to take 36 phase contrast images/well of C. acnes growth in a 24-well microplate every two hours. These time resolved images of C. acnes growth were saved and used by the system to calculate the % confluence metric of C. acnes growth over time. Representative images of C. acnes growth at designated time points are shown in FIG. 1C.

### Results

As shown in FIG. 1A, automated direct microscopic imaging allows for a faster time-to-detection when compared to the BacT/ALERT system, which is an automated rapid sterility test already validated for a 7-Day CAR-T Final Product Sterility Test Release. For media samples spiked with low levels of designated test microorganisms, the IncuCyte system's direct microscopic determination of increasing % confluence was faster than the BacT/ALERT time-to-detection based on the indirect colorimetric detection of CO₂ production in sample media bottles. The slow growing aerotolerant microorganism Cutibacterium acnes is one of the challenge microorganisms that limited the BacT/ALERT system to a 7-Day sterility test release time because of the time required to detect this microorganism in BacT/ALERT anaerobic sample bottles following low population count spike and recovery validation studies. As shown in FIGS. 1B and 1C, automated direct microscopic imaging allowed for the detection of C. acnes spiked into FTM at a population count of approximately 3 CFU/mL within 36 hours of incubation at 37 °C. In a separate BacT/ALERT study C. acnes (formerly Propionibacterium acnes) spiked into an anaerobic BacT/ALERT sample bottles at approximately 2 CFU/mL contained a time-to-detection of 94.3 hours based on the indirect colorimetric detection of CO₂ production in a sample media bottle, supporting the faster time-to-detection of direct microscopic imaging of this slow growing microorganism. Based on the faster time-to-detection comparison ratio automated direct cell imaging could realize a conservative 3-day sterility test release time compared to the current 7-day BacT/ALERT sterility test, with the potential for a <3-day release test to be validated and implemented.

In addition, the presence/absence screening of non-sterile product can also be achieved by performing a product dilution (i.e. 10-fold) and separating the contents into a bioburden sample and a 10 gram enrichment sample. The absence of growth in a 10-fold diluted bioburden sample demonstrates <10 CFU/g or mL in the original product sample. The IncuCyte system with its multiple fluorescent channels has the unique ability to develop applications for the rapid presence/absence screening of specified organisms using fluorescently labelled primary or secondary antibodies for quicker product release without the requirement for re-streaking onto selective media for identification of objectionable organisms like described in USP<62> Microbiological Examination of Nonsterile Products: Tests For Specified Microorganisms The capability of mammalian cell imaging system's for bioburden enumeration of nonsterile products following USP<62> could be realized by sample filtration onto a translucent membrane, either free-standing or in a filter plate format, followed by media sample incubation until the 10 micron system size limitation is achieved. Applications are currently available that would allow for the entire imaging of the translucent sample membrane surface for microorganism microcolony quantification. The mammalian cell imaging system fluorescent channels could also enable the development of multiplexed assays for mammalian cell viable count determination and microbial presence/absence contamination screening in the same bioreactor sample.

### Example 2: IncuCyte CAR-T Microbial Screening

### Materials and Methods

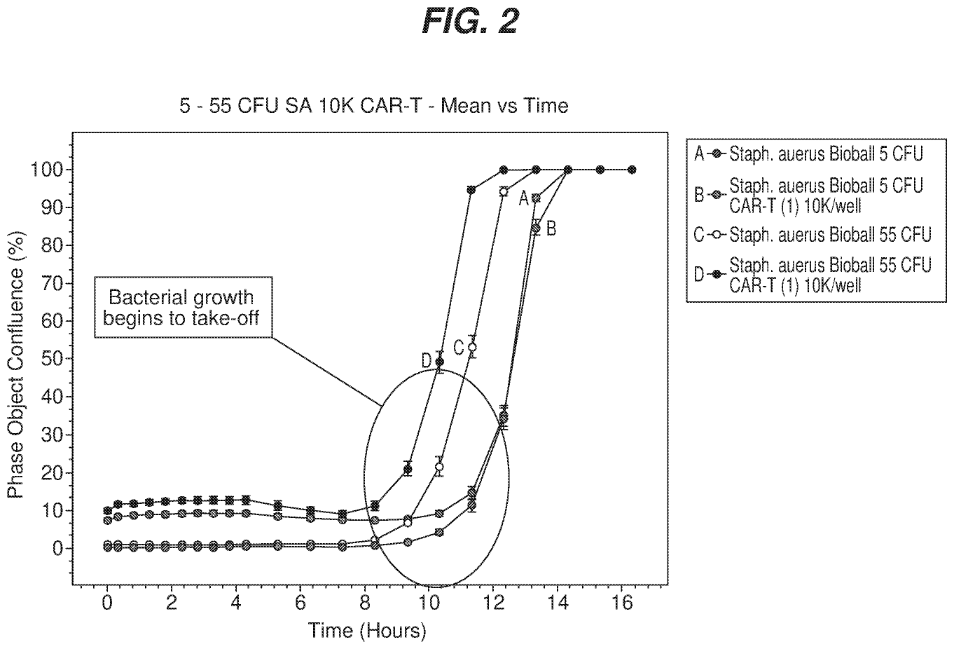

Staphylococcus aureus (SA) Bioballs™ (bioMerieux, USA) were resuspended following manufacturer's instructions. Two starting population counts (about 5 and 55 CFU) were then inoculated into duplicate individual wells of a 24-well cell culture plate (plasma treated) containing approximately 3 mL of TSB with and without approximately 10,000 healthy T cell donor CAR-T cells. The approximate 10,000 CAR-T cells were aliquoted from 1-ml CAR-T samples (LCAR+Healthy Human Donor T Cells, ~1E6/mL) Phase contrast imaging, 20x, 36 images/well was performed every hour. The time resolved images captured by the IncuCyte system were then used to calculate the % confluence metric to determine the time-to-detection based on an increase in % confluence. The results are shown in FIG. 2.

### Results

FIG. 2 illustrates the IncuCyte CAR-T SA microbial screening results. The graph shows a time course showing changes in % confluence over time across the different sample groups. The 5 CFU and 55 CFU SA inoculums spiked into 10,000 CAR-T cells were both detected within 12 hours of incubation at 37 °C based on the IncuCyte imaging software tracking a change in % confluence. As expected the sample wells containing the CAR-T cells have a higher starting % confluence value for both SA inoculum counts and the samples containing the higher SA starting population counts have a slightly faster time-to-detection based on the change in % confluence.

### Example 3: CHO Cell Bacillus subtilis Spore Spiking % Confluence Detection

### Materials and Methods

Bacillus subtilis spore Bioballs™ (bioMerieux, USA) were resuspended following manufacturer's instructions. A 6-well cell culture plate (plasma treated) containing approximately 8-mL of TSB in each well was inoculated with 1×10⁶ CHO (Chines Hamster Ovary) cells per well. Each well was then inoculated with approximately 25 CFU of B. subtilis spores with the exception of one well which was left as a CHO cell only baseline control. The plate was placed inside the IncuCyte system contained within a 37 °C Incubator and automated imaging was carried out every 30 minutes using a 20x objective.

### Results

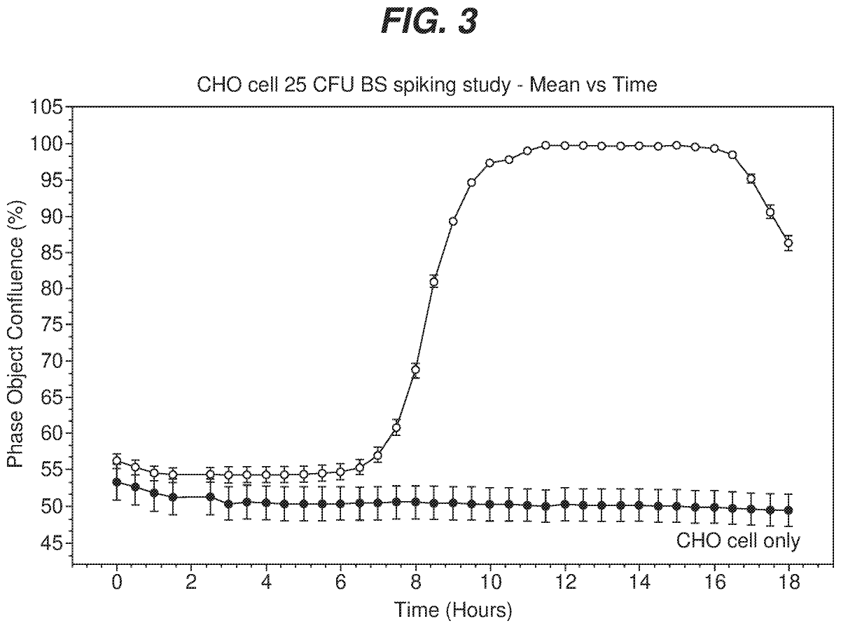

FIG. 3 illustrates CHO cell Bacillus subtilis spore spiking % confluence detection. The graph shows a time course showing detection of CHO cells contaminated with Bacillus subtilis spores. The presence of low levels of B. subtilis spores (25 CFU/well) was detected within 8 hours based on a chance in % confluence. The results can be confirmed visually, and the automated imaging is non-destructive allowing for the identification of any contaminants detected. These results suggest that the IncuCyte system could be used in place of conventional plating for rapid in-process monitoring of mammalian cell bioreactors. The system could be placed on the processing floor for at-line testing and the rapid results could be utilized to prevent the pooling of contaminated bioreactors during the manufacturing process, resulting in significant cost savings.

### Example 4: Staphylococcus epidermidis (SE) Prebiotic and Antimicrobial Screening

### Materials and Methods

A stationary phase Staphylococcus epidermidis (SE) culture grown in TSB was diluted in TSB to obtain a population count of approximately 2000 CFU per mL. Ten microliter aliquots (approximately 25 CFU) of this SE suspension were then inoculated into each designated well of a 24 well cell culture plate (plasma treated) containing 1% soluble ingredient suspensions in approximately 1 mL of TSB. Each soluble ingredient was filter sterilized using a 0.22 μm filter membrane prior to making the 1% test suspensions. Designated wells containing soluble ingredient only and SE only were included as negative and positive controls respectively. The sample plate was then placed into an IncuCyte system contained within a 37 °C incubator and imaging was automatically carried out every hour using a 20x objective.

### Results

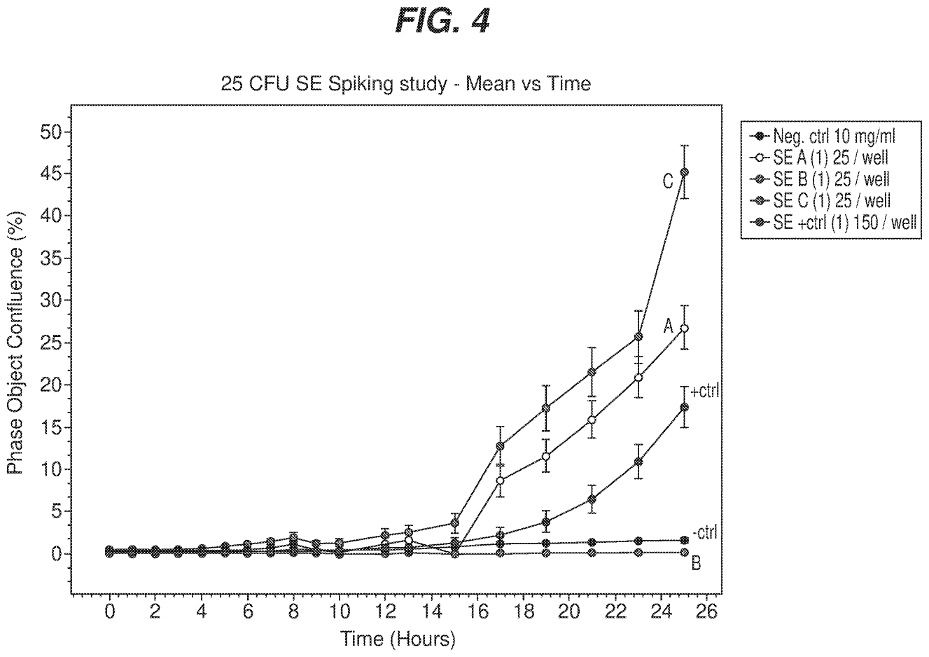

FIG. 4 illustrates Staphylococcus epidermidis (SE) Prebiotic and Antimicrobial Screening (1%). SE Prebiotic activity of Ingredient A (sterile filtered) was detected based on the faster % confluence growth compared to the positive control containing only SE in TSB. SE Antimicrobial activity of Ingredient B (sterile filtered) was detected by no change in % confluence growth over time. SE Prebiotic activity of Ingredient C (sterile filtered) was detected by faster % confluence growth compared to positive control. Of all ingredients, only negative controls were negative for growth.

### Example 5: Natural Product Extract Staphylococcus aureus (SA) Antimicrobial Screening

### Materials and Methods

A natural product extract library (Phytotitre, Caithness Biotechnologies (Leicester, United Kingdom), containing 50 μL individual sample aliquots (10 mg/mL [1% starting concentration in DMSO]) in a 96 well cell culture plate was screened for the presence of SA antimicrobial using the IncuCyte system. To make the SA microbial test suspension a stationary phase SA culture grown in TSB was diluted in TSB to obtain a population count of approximately 3E6 CFU per mL. Ten μL of each designated natural product extract was then mixed with 90 μL of the SA TSB suspension in individual wells of a new 96 well cell culture plate (plasma treated) to inoculate 0.1% of the natural product extract samples with approximately 20,000 CFU of SA per well. Three SA positive control wells containing 90 μL of the SA TSB suspension (approximately 2000 CFU/well) only were included in addition to 3 DMSO controls containing 90 μL of the SA TSB suspension (approximately 2000 CFU/well) and 10 μL of DMSO only. The cell culture plate samples were then placed inside the IncuCyte system at 37 °C and automatically imaged every hour with a 20x objective.

### Results

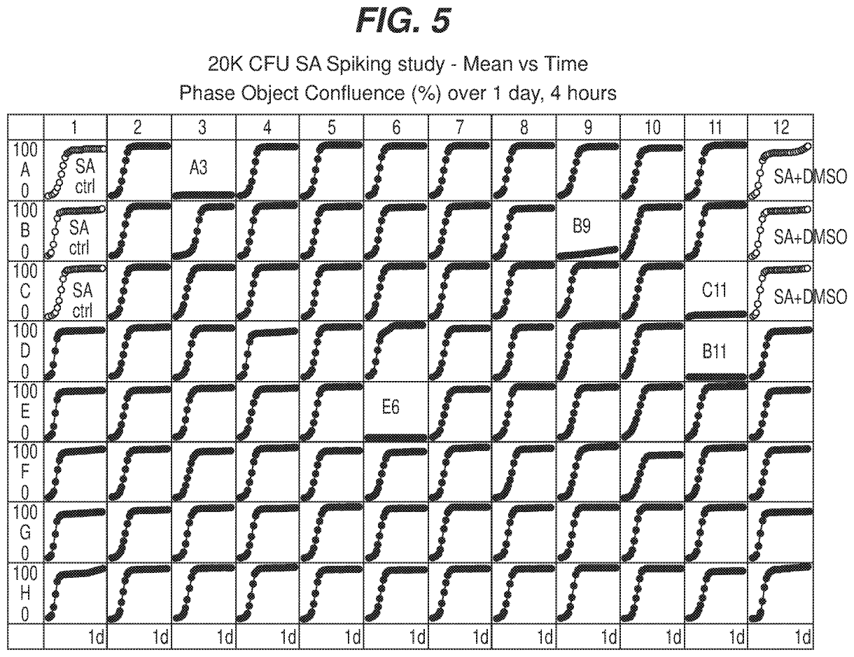

FIG. 5 illustrates a Natural Product Extract Staphylococcus aureus (SA) Antimicrobial Screening (0.1%). 96-well Natural Product Extract Microplate % Confluence Results Map. Natural Product Extract SA Antimicrobial activity was detected by no change in % confluence growth over time for sample wells A3, E6, B9, C11 and D11. The SA positive controls with and without DMSO contained representative positive growth verifying that the presence of DMSO (10% concentration) in the sample wells did not inhibit SA microbial growth and that the antimicrobial screening results were due to the natural product extracts present in the test sample wells.

### Embodiments

The following exemplary embodiments further describe optional aspects of the presently disclosed technology and are part of the Detailed Description. These examplary embodiments are set forth in a format substantially akin to claims (each with numerical designations followed by a capital letter), although they are not technically claims of the present application. The following exemplary embodiments refer to each other in dependent relationships as “embodiments” instead of “claims.”

1A. A method for detecting a microbe in a sample comprising: detecting confluence of the sample.

2A. The method of embodiment 1A, wherein the confluence is detected using an image-based metric.

3A. The method of embodiment 2A, wherein the image-based metric comprises direct imaging.

4A. The method of embodiment 3A, wherein the direct imaging is direct cell imaging.

5A. The method of any one of embodiments 1A-4A, wherein the sample is in an incubator.

6A. The method of any one of embodiments 1A-5A, wherein the detecting comprises detecting using a microscope or image sensor.

7A. The method of any one of embodiments 1A-6A, wherein the confluence of the sample is compared to the confluence of a control sample.

8A. The method of any one of embodiments 1A-7A, wherein the confluence of the sample is monitored over a period of time.

9A. The method of any one of embodiments 1A-8A, wherein the sample has been contacted with an antibody to the microbe.

10A. The method of any one of embodiments 1A-9A, wherein the sample has been contacted with a fluorescent agent or a bioluminescent agent.

11A. The method of any one of embodiments 1A-10A, further comprising detecting fluorescence or bioluminescence of the sample.

12A. The method of any one of embodiments 1A-11A, wherein the microbe is Cutibacterium acnes, Staphylococcus aureus, Aspergillus brasiliensis, Candida albicans, Bacillus subtilis, Escherichia coli, or Pseudomonas aeruginosa.

13A. The method of any one of embodiments 1A-12A, wherein the detecting confluence is automated.

14A. The method of any one of embodiments 1A-13A, wherein the confluence is percent confluence.

15A. A method for assaying a test agent comprising: adding a test agent to a sample comprising a microbe; and detecting confluence of the sample.

16A. The method of embodiment 15A, wherein the confluence is detected using an image-based metric.

17A. The method of embodiment 16A, wherein said image-based metric comprises direct imaging.

18A. The method of embodiment 17A, wherein said direct imaging is direct cell imaging.

19A. The method of any one of embodiments 15A-18A, wherein the sample is in an incubator.

20A. The method of any one of embodiments 15A-19A, wherein the detecting comprises detecting using a microscope or image sensor.

21A. The method of any one of embodiments 15A-20A, wherein the confluence of the sample is compared to the confluence of a control sample.

22A. The method of any one of embodiments 15A-21A, wherein the confluence of the sample is monitored over a period of time.

23A. The method of any one of embodiments 15A-22A, wherein the test agent is a compound, a supplement, an antibiotic, a bacteriophage, or a combination thereof.

24A. The method of any one of embodiments 15A-23A further comprising identifying the test agent as a prebiotic when the confluence of the sample is greater than the confluence of the control sample.

25A. The method of any one of embodiments 15A-23A further comprising identifying the test agent as a prebiotic when the confluence of the sample increases over a period of time.

26A. The method of any one of embodiments 15A-23A further comprising identifying the test agent as an antimicrobial agent when the confluence of the sample is less than the confluence of the control sample.

27A. The method of any one of embodiments 15A-24A further comprising identifying the test agent as an antimicrobial agent when the confluence of the sample decreases over a period of time.

28A. The method of any one of embodiments 15A-27A, wherein the sample has been contacted with an antibody to the microbe.

29A. The method of any one of embodiments 15A-28A, wherein the sample has been contacted with a fluorescent agent or a bioluminescent agent.

30A. The method of any one of embodiments 15A-29A, further comprising detecting fluorescence or bioluminescence of the sample.

31A. The method of any one of embodiments 15A-30A, wherein the microbe is Cutibacterium acnes, Staphylococcus aureus, Aspergillus brasiliensis, Candida albicans, Bacillus subtilis, Escherichia coli, or Pseudomonas aeruginosa.

32A. The method of any one of embodiments 15A-31A, wherein the detecting confluence is automated.

33A. The method of any one of embodiments 15A-32A, wherein the confluence is percent confluence.

### References

ASM Minireview, Sterility Testing for Cellular Therapies: What Is the Role of the Clinical Microbiology Laboratory. 2020. Journal of Clinical Microbiology Vol. 58 Issue 7 Pages 1-14.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for detecting a microbe in a sample comprising:
detecting confluence of the sample.

2. The method of claim 1, wherein the confluence is detected using an image-based metric.

3. The method of claim 2, wherein the image-based metric comprises direct imaging.

4. The method of claim 3, wherein the direct imaging is direct cell imaging.

5. The method of claim 1, wherein the sample is in an incubator.

6. The method of claim 1, wherein the detecting comprises detecting using a microscope or image sensor.

7. The method of claim 1, wherein the confluence of the sample is compared to the confluence of a control sample.

8. The method of claim 1, wherein the confluence of the sample is monitored over a period of time.

9. The method of claim 1, wherein the sample has been contacted with an antibody to the microbe.

10. The method of claim 1, wherein the sample has been contacted with a fluorescent agent or a bioluminescent agent.

11. The method of claim 1, further comprising detecting fluorescence or bioluminescence of the sample.

12. The method of claim 1, wherein the microbe is Cutibacterium acnes, Staphylococcus aureus, Aspergillus brasiliensis, Candida albicans, Bacillus subtilis, Escherichia coli, or Pseudomonas aeruginosa.

13. The method of claim 1, wherein the detecting confluence is automated.

14. The method of claim 1, wherein the confluence is percent confluence.

15. A method for assaying a test agent comprising:
adding a test agent to a sample comprising a microbe; and
detecting confluence of the sample.

16. The method of claim 15, wherein the confluence is detected using an image-based metric.

17. The method of claim 16, wherein said image-based metric comprises direct imaging.

18. The method of claim 17, wherein said direct imaging is direct cell imaging.

19. The method of claim 15, wherein the sample is in an incubator.

20. The method of claim 15, wherein the detecting comprises detecting using a microscope or image sensor.

21. The method of claim 15, wherein the confluence of the sample is compared to the confluence of a control sample.

22. The method of claim 15, wherein the confluence of the sample is monitored over a period of time.

23. The method of claim 15, wherein the test agent is a compound, a supplement, an antibiotic, a bacteriophage, or a combination thereof.

24. The method of claim 15 further comprising identifying the test agent as a prebiotic when the confluence of the sample is greater than the confluence of the control sample.

25. The method of claim 15 further comprising identifying the test agent as a prebiotic when the confluence of the sample increases over a period of time.

26. The method of claim 15 further comprising identifying the test agent as an antimicrobial agent when the confluence of the sample is less than the confluence of the control sample.

27. The method of claim 15 further comprising identifying the test agent as an antimicrobial agent when the confluence of the sample decreases over a period of time.

28. The method of claim 15, wherein the sample has been contacted with an antibody to the microbe.

29. The method of claim 15, wherein the sample has been contacted with a fluorescent agent or a bioluminescent agent.

30. The method of claim 15, further comprising detecting fluorescence or bioluminescence of the sample.

31. The method of claim 15, wherein the microbe is Cutibacterium acnes, Staphylococcus aureus, Aspergillus brasiliensis, Candida albicans, Bacillus subtilis, Escherichia coli, or Pseudomonas aeruginosa.

32. The method of claim 15, wherein the detecting confluence is automated.

33. The method of claim 15, wherein the confluence is percent confluence.
